(19) United States
(12) Patent Application Publication
(10) Pub. No.: US 2007/0060975 A1
(43) Pub. Date: Mar. 15, 2007

(54) Combination electrode-battery and programming assembly for a miniature wireless transcutaneous electrical neuro or muscular-stimulation unit

(76) Inventors: Jeffrey S. Mannheimer, Princeton, NJ (US); Barry Sauls, Crystal River, FL (US)

Correspondence Address: RANDALL B. BATEMAN;BATEMAN IP LAW GROUP, 8 EAST BROADWAY, SUITE 550, PO BOX 1319, SALT LAKE CITY, UT, 84110, US

(21) Appl. No.: 11/434,453

(22) Filed: May 15, 2006

Related U.S. Application Data
(63) Continuation-in-part of application No. 10/208,223, filed on Jul. 30, 2002, now abandoned.
Application No. 10/208,223 is a continuation-in-part of application No. 09/350,426, filed on Jul. 8, 1999, now Pat. No. 6,445,955.

(51) Int. Cl. A61N 1/34

(52) U.S. Cl. 607/46; 607/2

(57) ABSTRACT

A flexible circuit combination electrode-battery assembly for a transcutaneous electrical neuro or muscular stimulation unit is provided, which is capable of being removably attached to both the patient and the transcutaneous electrical neuro or muscular stimulation unit. The assembly is generally comprised of two sided electrodes imprinted on a flexible, non-conductive substrate, and batteries for providing power. The design relies on a conductive via or holes in the non-conductive substrate filed with a conductive material to transfer current from one side of the non conductive substrate to the other. An optional resistor may be included to allow the electrode assembly to program a TENS device. An electrically conductive hydrogel or conductive adhesive is provided for attachment to the patient and for ensuring the integrity of the electrical contact with the patient.

### CLAIM OF PRIORITY

This application is a continuation-in-part of U.S. application Ser. No. 10/208223 filed on Jul. 30, 2002 which is a continuation-in-part of U.S. application Ser. No. 09/350,426, filed on Jul. 8, 1999, the contents of which are incorporated herein in their entirety by reference.

### TECHNICAL FIELD

The invention relates generally to transcutaneous electrical neuro-stimulation (TENS) units and this invention particularly relates to an electrode-battery assembly for a miniaturized wireless TENS unit capable of being pre-programmed to achieve a variety of waveforms, with or without the use of a remote controller means, each waveform having unique features capable of masking pain or promoting functional restoration in a user's body.

### BACKGROUND OF THE INVENTION

TENS devices have been traditionally prescribed in the medical industry for chronic pain. While patients experiencing acute pain are prescribed anti-inflammatory and narcotic agents, the treatment of chronic pain, usually defined as unrelieved pain for at least 30 days, can also be dealt with via TENS-related prescriptions, as a non-medicinal alternative. However, TENS devices have been shown to provide rapid and effective relief for acute pain without side effects or the possibility of addiction. TENS does not utilize anesthesia or narcosis. Patients remain awake, alert and functional, and retain the protective qualities of increased pain perception.

TENS is commonly used for acute pain management by physical therapists in comprehensive rehabilitation programs in conjunction with other treatments. TENS devices are usually large as well as being complex, expensive and require lead wires running to each electrode, making them difficult for use at home, at work or at play.

Previous attempts have been made to design improved electrotherapy devices, certain features of which are generally described in U.S. Pat. No. 5,620,470 to Gliner et al.; U.S. Pat. No. 5,607,454 to Cameron et al.; U.S. Pat. No. 5,601,612 to Gliner et al.; U.S. Pat. No. 5,593,427 to Gliner et al.; U.S. Pat. No. 5,584,863 to Rauch et al.; U.S. Pat. No. 5,578,060 to Pohl et al.; U.S. Pat. No. 5,573,552 to Hansjurgens; U.S. Pat. No. 5,549,656 to Reiss; U.S. Pat. No. 5,514,165 to Malaugh et al.; U.S. Pat. No. 5,476,481 to Schondorf; U.S. Pat. No. 5,387,231 to Sporer; U.S. Pat. No. 5,397,338 to Grey et al.; U.S. Pat. No. 5,374,283 to Flick; U.S. Pat. No. 5,354,320 to Schaldach et al.; U.S. Pat. No. 5,304,207 to Stromer; U.S. Pat. No. 5,183,041 to Toriu et al.; U.S. Pat. No. 4,989,605 to Rossen; U.S. Pat. No. 4,759,368 to Spanton et al.; U.S. Pat. No. 4,699,143 to Dufresne et al.; and U.S. Pat. No. 4,398,545 to Wilson, all of which are incorporated herein by reference.

The '470 patent to Gliner et al. describes an external defibrillator and defibrillation method that automatically compensates for patient-to-patient impedance differences in the delivery of electrotherapeutic pulses for defibrillation and cardioversion. In a preferred embodiment, the defibrillator has an energy source that may be discharged through electrodes on the patient to provide a biphasic voltage or current pulse. In one aspect of the invention, the first and second phase duration and initial first phase amplitude are predetermined values. In a second aspect of the invention, the duration of the first phase of the pulse may be extended if the amplitude of the first phase of the pulse fails to fall to a threshold value by the end of the predetermined first phase duration, as might occur with a high impedance patient. In a third aspect of the invention, the first phase ends when the first phase amplitude drops below a threshold value or when the first phase duration reaches a threshold time value, whichever comes first, as might occur with a low to average impedance patient. This method and apparatus of altering the delivered biphasic pulse thereby compensates for patient impedance differences by changing the nature of the delivered electrotherapeutic pulse, resulting in a smaller, more efficient and less expensive defibrillator.

The '454 patent to Cameron et al. describes an electrotherapy method and apparatus for delivering a multiphasic waveform from an energy source to a patient. The preferred embodiment of the method comprises the steps of charging the energy source to an initial level; discharging the energy source across the electrodes to deliver electrical energy to the patient in a multiphasic waveform; monitoring a patient-dependent electrical parameter during the discharging step; shaping the waveform of the delivered electrical energy based on a value of the monitored electrical parameter, wherein the relative duration of the phases of the multiphasic waveform is dependent on the value of the monitored electrical parameter. The preferred apparatus comprises an energy source; two electrodes adapted to make electrical contact with a patient; a connecting mechanism forming an electrical circuit with the energy source and the electrodes when the electrodes are attached to a patient; and a controller operating the connecting mechanism to deliver electrical energy from the energy source to the electrodes in a multiphasic waveform, the relative phase durations of which are based on an electrical parameter monitored during delivery of the electrical energy. The preferred defibrillator apparatus weighs less than 4 pounds and has a volume less than 150 cubic inches, and most preferably, weighs approximately three pounds or less and has a volume of approximately 141 cu. in.

The '612 patent to Gliner et al. describes an external defibrillator and defibrillation method that automatically compensates for patient-to-patient impedance differences in the delivery of electrotherapeutic pulses for defibrillation and cardioversion. In a preferred embodiment, the defibrillator has an energy source that may be discharged through electrodes on the patient to provide a biphasic voltage or current pulse. In one aspect of the invention, the first and second phase duration and initial first phase amplitude are predetermined values. In a second aspect of the invention, the duration of the first phase of the pulse may be extended if the amplitude of the first phase of the pulse fails to fall to a threshold value by the end of the predetermined first phase duration, as might occur with a high impedance patient. In a third aspect of the invention, the first phase ends when the first phase amplitude drops below a threshold value or when the first phase duration reaches a threshold time value, whichever comes first, as might occur with a low to average impedance patient. This method and apparatus of altering the delivered biphasic pulse thereby compensates for patient impedance differences by changing the nature of the delivered electrotherapeutic pulse, resulting in a smaller, more efficient and less expensive defibrillator.

The '427 patent to Gliner et al. describes an external defibrillator and defibrillation method that automatically compensates for patient-to-patient impedance differences in the delivery of electrotherapeutic pulses for defibrillation and cardioversion. In a preferred embodiment, the defibrillator has an energy source that may be discharged through electrodes on the patient to provide a biphasic voltage or current pulse. In one aspect of the invention, the first and second phase duration and initial first phase amplitude are predetermined values. In a second aspect of the invention, the duration of the first phase of the pulse may be extended if the amplitude of the first phase of the pulse fails to fall to a threshold value by the end of the predetermined first phase duration, as might occur with a high impedance patient. In a third aspect of the invention, the first phase ends when the first phase amplitude drops below a threshold value or when the first phase duration reaches a threshold time value, whichever comes first, as might occur with a low to average impedance patient. This method and apparatus of altering the delivered biphasic pulse thereby compensates for patient impedance differences by changing the nature of the delivered electrotherapeutic pulse, resulting in a smaller, more efficient and less expensive defibrillator.

The '863 patent to Rauch et al. describes a system for tissue-impedance matched pulsed radio frequency (PRF) electrotherapy, which includes a power supply, an excitation board for generating PRF signals of a selectable frequency, the board having an input from the power supply. The system also includes a power amplifier for signals from the excitation board. Included is a subsystem for controlling pulse width duration, pulse burst repetition rate, and amplitude of the PRF signals, the controlling system having an input from the power supply. Further provided is a subsystem for continually comparing the amplitude of the PRF signals outputted from the amplifier to a reference value, therefore including a feedback circuit responsive to information between the compared signals and the reference value, that is inputted to the controlling subsystem for adjustment of the amplitude and impedance of the PRF signals from the excitation board, and a comparing system that also includes an output of power and impedance compensated PRF signals. The system also includes a variable reactance therapeutic applicator having, as a coaxial cable input, the power and impedance compensated PRF signals outputted from the comparing subassembly, the applicator including a treatment surface having an effective physiologic impedance in the range of 25 to 75 ohms.

The '060 patent to Pohl et al. describes a reconfigurable physical therapy apparatus and a method of providing operator-selected stimuli to a patient are provided. The apparatus preferably has a physical therapy applicator including a transducer for applying a therapeutic treatment to a patient, and a memory for storing identification data representative of a plurality of physical ailments for each of a plurality of human body areas and a set of transducer operational parameters associated with each predetermined physical ailment and each predetermined body area. The apparatus also has an ailment display screen responsive to the memory device for displaying at least one of the identification data representative of a plurality of physical ailments, which are associated with at least one of the identified human body areas. An ailment selector is positioned in electrical communication with at least the memory device and being responsive to operator selection of one of the identified physical ailments, which are associated with human body areas for obtaining the associated transducer operational parameters. The apparatus further has a transducer reconfigurer positioned in electrical communication with the transducer of the applicator and being responsive to the ailment selector for reconfiguring the transducer to provide therapeutic treatment to the identified body part according to the obtained transducer operational parameters

The '552 patent to Hansjurgens describes an apparatus for electrotherapeutic applications operating in the medium-frequency range between 1000 Hz and 100,000 Hz where, in relation to a body part to be treated, a circuit with medium-frequency current (MF current) is applied across two electrodes, the invention proposes to keep the amplitude of the MF current constant and to modulate the frequency by one thousand to several thousand Hz (corner frequencies) with a modulation frequency of >0 to several hundred Hz (for instance 200 Hz) in order to generate in synchronism with the modulation frequency action potentials in the treatment area.

The '656 patent to Reiss describes a combined dual channel electromuscular stimulator for directing electrical pulses into the skin and a dual channel electromyograph for detecting electrical signals generated in muscles. The electromuscular stimulator includes electronic circuitry for generating electrical pulses, controlling the pulse rate and intensity and controlling various pulse characteristics. The pulses are administered by skin contacting electrodes. The electromyograph includes skin contacting electrodes for receiving input signals from the skin and electronic circuitry for receiving detected signals without interference with the stimulator output signals, amplifying, filtering and displaying the input signals. A control panel includes switches and controls for varying the various system parameters.

The '165 patent to Malaugh et al. describes an electrotherapy stimulation unit having a high voltage pulsed current (HVPC) electrotherapy stimulation device providing short duration low amperage high voltage constant charge HVPC pulses to a patient to reduce pain, and a neuromuscular stimulation (NMS) electrotherapy device providing constant current NMS pulses to a patient to re-educate and prevent atrophy of muscle tissue. The HVPC device has a voltage source and at least one HVPC output circuit having a coil, a switching device, and a holding capacitor. When the switching device is turned on, an increasing current is drawn through the coil. When the switching device is turned off, a voltage spike results across the coil, charging the holding capacitor. Thereafter, the charge dissipates into the patient. The HVPC device senses the voltage provided by the voltage source and calculates the period of time the switching device is turned on based upon the sensed voltage and the pre-selected peak voltage of the voltage spike. The HVPC device provides a train of HVPC pulses, each HVPC pulse comprising first and second voltage spikes. The HVPC device detects whether a patient is properly connected to the HVPC output of the output circuit. If the second voltage spike is larger than the first by a predetermined value, a patient is not connected to the HVPC output circuit, and the output circuit is disabled.

The '481 patent to Schondorf describes an electrotherapeutic field stimulator that includes a pair of electrodes for applying the electricity to the body in the form of an electric field and a generator for providing the electricity to the electrodes in the form of at least two superimposed alternating current fields of different frequencies to provide the treatment waveform.

The '231 patent to Sporer describes a method of microcurrent electrotherapy utilizing a combination of specified values for selected parameters including electrical stimulus wave form, direction, magnitude, voltage, polarity and frequency to provide a variety of therapeutic enhancements.

The '338 patent to Grey et al. describes an electrotherapy device for delivering electrical energy to subcutaneous, excitable tissues in and around the joints of the human body for the purposes of pain control and the promotion of tissue healing post-injury is provided. The device includes a housing containing at least one pair of electrodes connected to an electronics unit. The device is specifically designed to be small, portable and lightweight so as to not interfere with user movements and/or function. The electronics unit consists of a housing that contains batteries, a microcontroller integrated circuit (including associated control software) coupled to a transistor-based intensity stage, which is then coupled to a transformer-based output stage coupled to subminiature jacks used to connect the electronics unit to the electrodes. Control software monitors user-controlled mechanical switches for the selection of one of six operational modes (TENS, MENS, or iontophoresis) and one of six discrete intensity levels within each operational mode. The housing is a flexible, elastic sleeve that conforms to joint anatomy and has the electrodes sewn into specific positions such that when the user puts on the sleeve, the electrodes are placed at the correct anatomic position over the affected joint.

The '283 patent to Flick describes an electrical therapeutic apparatus (10) for the treatment of body pain and edema. The apparatus has an electrical pulse-producing device (11) coupled to wrap (12) by conductor (13). The wrap is comprised of nylon coated with silver, which forms an electrode. A second electrode (14) is coupled by conductors (15) to the device.

The '320 patent to Schaldach et al describes a neurostimulator for generating stimulation pulses for the central or peripheral nervous system, particularly against pain in the region of the spinal cord and includes a control circuit for generating stimulation pulses with a pulse generator whose output is connected with stimulation electrodes. The stimulation pulses are generated at periodic intervals with an activity period corresponding essentially to an effective duration corresponding to a biological half-lifetime of a body's own active substances. The control circuit creates a respective rest period corresponding to a time required by the body's own active substances to regenerate themselves for a corresponding activity period.

The '207 patent to Stromer describes an improved electro-stimulator apparatus, comprising first and second electrodes spaced-apart at a predetermined distance, an electrical signal generator for providing pulses of predetermined width and repetition rate to the spaced-apart electrodes, and an LED providing a beam of light projecting between the spaced-apart electrodes toward the object intended to be electro-stimulated. The electrodes have substantially co-planar external faces approximately perpendicular to the light beam. The electrodes, signal generator and LED are mounted in an elongated housing having a longitudinal central axis. The electrodes are exposed on an end and the light beam is emitted from the same end and substantially parallel to the central axis. An ON/OFF switch actuates the signal generator and the LED when turned ON. It automatically turns OFF state when released so that the signal generator and the LED are always ON or OFF together.

The '041 patent to Toriu et al. describes a transcutaneous electric nerve stimulator having a plurality of treatment modes and producing a low-frequency pulse of a frequency corresponding to a selected treatment mode. A plurality of indicators is provided in association with the respective treatment modes such that one of the indicators corresponding to the selected treatment mode is caused to blink in synchronism with the produced low-frequency pulse.

The '605 patent to Rossen describes an improved transcutaneous electrical nerve stimulator (TENS) involving a microcurrent (typically 25 to 900 microamps) D.C. carrier signal (typically 10,000 to 19,000 Hz, preferably 15,000 Hz) that is modulated on and off in time (typically at 0.3 Hz up to 10,000 Hz, preferably 9.125 Hz followed by 292 Hz) and further inverted about every second by reversing the polarity of the signal at the electrodes. Such a device has been found to be useful in alleviating pain very rapidly.

The '368 patent to Spanton et al. describes a transcutaneous nerve stimulating device having a plurality of operating modes, namely burst, normal (single amplitude/single pulse width), rate modulation, amplitude modulation and strength-duration/rate modulation. In the lattermost mode, the rate modulation control circuitry acts independently of the inter-related amplitude and pulse width modulations to result in a means of nerve stimulation obviating the phenomenon of accommodation.

The '143 patent to Dufresne et al. describes an electrical stimulator for biological tissue having remote control. A remote element communicates an operator response to the electrical stimulator. A control element samples the communication from the remote element and adjusts one or more of certain sets of stimulus parameters maintained in a storage element and utilizes the adjusted stimulus parameters to generate an electrical stimulus signal or utilizes the communication from the remote element to trigger the generation of an electrical stimulus signal based upon the stored stimulus parameters.

The '545 patent to Wilson describes a bandage to be applied adjacent to an injured portion of a patient's body that contains electronic circuitry which delivers electric pulses into the body to block or mask the pain arising from the injury. The bandage includes an inner unit adapted to be applied directly onto the patient's skin and an outer unit adapted to be removably applied upon the inner unit. The inner unit includes spaced apart conductive portions, which contact the patient's skin. The outer unit includes a power source and an electronic circuit, which applies a voltage output to the conductive portions of the inner unit. The voltage output is transmitted through the conductive portions to the patient's skin to cause low current electrical pulses within the patient's body to block or mask the pain arising from the injury.

However, none of these references, either alone or in combination with others, describes a miniature, wireless transcutaneous neuro stimulation device with or without a remote controlled configuration that has pre-programmable waveform modes and includes a unique detachable electrode-battery assembly.

Consequently there is a need in the art for a combination electrode-battery assembly for a miniaturized, wireless TENS device that can be utilized by the patient without the embarrassment of unsightly wires protruding through clothing

There is a further need in the art for such a device that can be placed on a variety of sites on the patient's body,

There is a further need in the art for such a device that can be virtually unseen.

There is a further need in the art for such a device that can be controlled by a controller means to transmit pulses at different intensities and frequencies adaptable to the patient's particular physical malady.

There is a further need in the art for a combination electrode-battery assembly for a miniature, wireless TENS-related device that can easily be programmed by the user, with or without the use of a remote controller,

There is a further need in the art for such a device that can provide a variety of waveforms at various programmable intensities to a number of pain sites on the user's body, and which

There is a further need in the art for such a device that can be easily adaptable for use with splints, braces and bandages.

There is a further need in the art for an electrode assembly capable of being made by conventional printing techniques.

There is a further need in the art for a self-programming TENS device capable of being used by a lay consumer.

### SUMMARY OF THE INVENTION

These needs are met by providing an electrode-battery assembly used in a miniature wireless transcutaneous electrical neuro or muscular-stimulation unit comprising a plurality of electrodes each having an internal and external side, at least one battery having a positive and negative pole, a flexible conductive carrier with a hydrogel, which carries current to a pain site or other area on a user's body via the electrodes, conductive film comprised of at least three current carrier runners, wherein two of the runners are in direct contact with each of the positive and negative poles of the battery to provide power to an electronics unit which provides the electrical stimulation to the electrode, and a third or more of said runners which are in direct contact with an output on the electronics unit and the hydrogel, and a mechanical means for securing the battery to the runners to the positive and negative battery poles.

In an alternate embodiment, the electrode-battery assembly is disposable and can be replaced upon depletion of the battery.

In another alternate embodiment, the conductive film of the electrode-battery assembly is comprised of a silver alloy film, a silver conductive ink channel or some other flexible low impedance material.

In another alternate embodiment, the external side of the electrode-battery assembly is covered by a molded cover comprised of a cosmetically appealing molded foam or elastomer.

In another alternate embodiment, the electrode-battery assembly is rechargeable.

In yet another embodiment, the electrode is manufactured from sheets of non-conductive substrate onto which conductors are applied.

Therefore, it is an object of the present invention to provide a combination electrode-battery assembly for a miniaturized, wireless TENS device that can be utilized by the patient without the embarrassment of unsightly wires protruding through clothing

It is a further object to provide a device that can be placed on a variety of sites on the patient's body,

It is a further object to provide a device that can be virtually unseen.

It is a further object to provide a device that can be controlled by a controller means to transmit pulses at different intensities and frequencies adaptable to the patient's particular physical malady.

It is a further object to provide a combination electrode-battery assembly for a miniature, wireless TENS-related device that can easily be programmed by the user, with or without the use of a remote controller,

It is a further object to provide a device that can provide a variety of waveforms at various programmable intensities to a number of pain sites on the user's body, and which

It is a further object to provide a device that can be easily adaptable for use with splints, braces and bandages.

It is a further object to provide a device that has easily replaceable electrodes.

It is a further object to provide a device with inexpensive electrodes.

It is a further object to provide a device with electrodes which are capable of programming the TENS device to deliver specific pre-set waveforms.

### BRIEF DESCRIPTION OF THE DRAWINGS

FIG. 1 shows an overhead view of the electrode-battery assembly 18.

FIG. 2 shows an end view of the electrode-battery assembly 18 of FIG. 1.

FIG. 3 shows a side view of the electrode-battery assembly 18 of FIG. 1.

FIGS. 4a and 4b show usage of a conductive adhesive.

FIG. 5a shows an assembled electrode battery assembly

FIG. 5b shows an exploded view of the layers of the electrode-battery assembly

FIG. 6 shows an overhead view of the electrode battery assembly

FIG. 7 shows an overhead close up of the individual components of the electrode-battery assembly.

### DETAILED DESCRIPTION OF THE PREFERRED EMBODIMENT

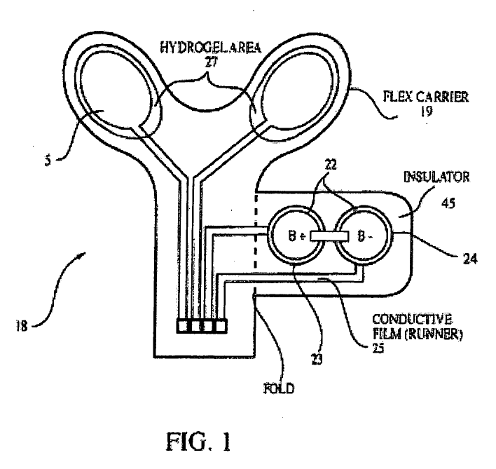

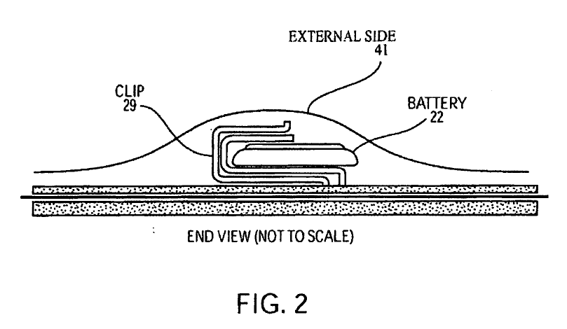

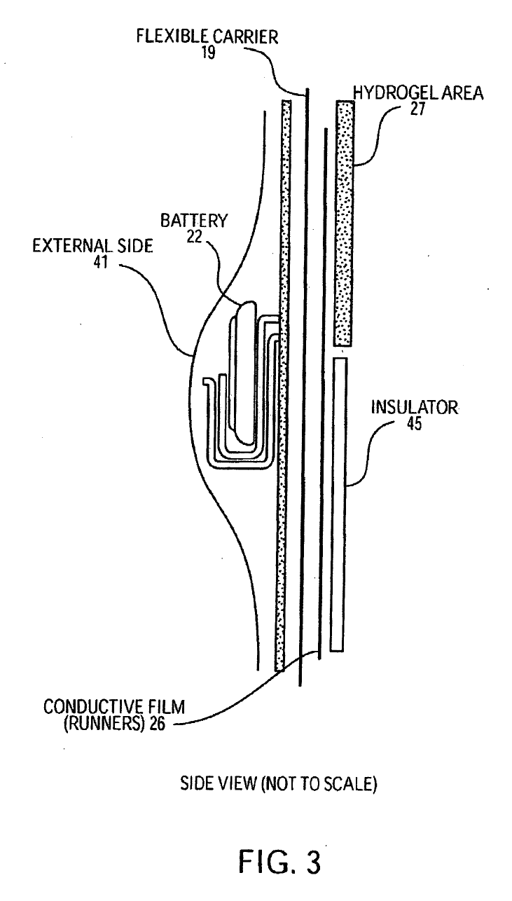

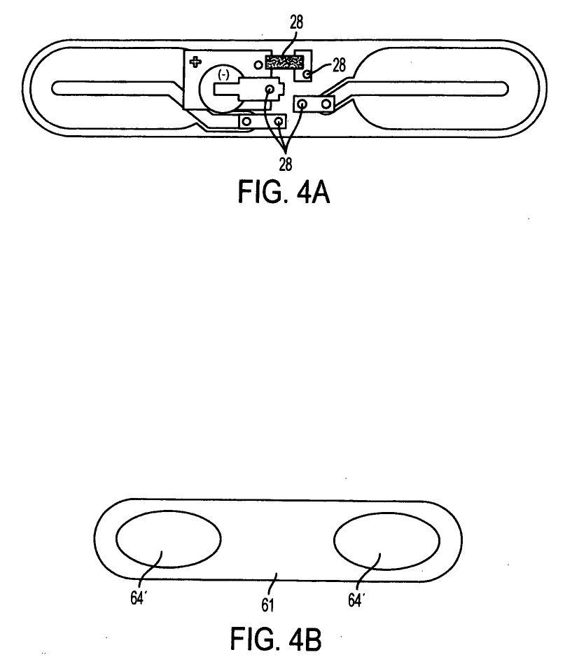

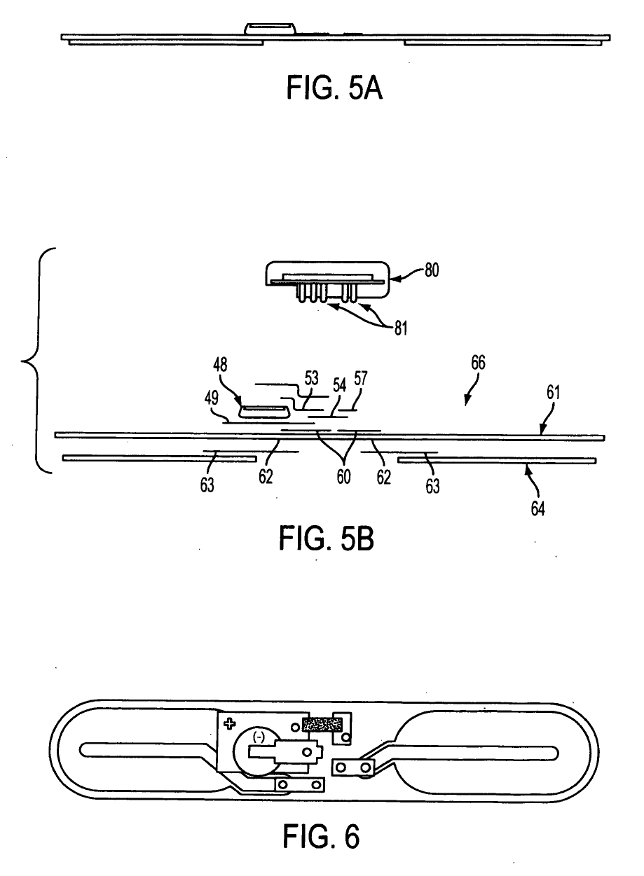

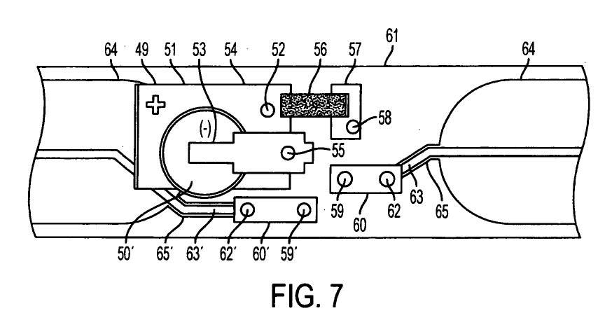

Turning now to the drawings, in which like numerals indicate like elements throughout the several views a disposable electrode-battery assembly 18, as seen in FIGS. 1, 2 and 3, resides within the housing 2 of the present invention. FIG. 1 shows the assembly 18 comprised of a plurality of electrodes 5 each having an internal and external side and a plurality of batteries 22 each having a positive pole 23 and a negative pole 24. Current carrying runners 25 comprise a conductive film 26. Two of these runners 25 make direct contact to the positive 23 and negative 24 poles of the battery 22, while the third makes contact with conductive hydrogel 27, which carries the stimulating current to the patient via each electrode 5. Contact to the battery poles is secured either by a conductive adhesive 28 as seen in FIG. 4 or a mechanical clip 29 as seen in FIG. 2. in order to apply the required pressure. The conductive film 26 may be a silver alloy film or other flexible low impedance material. The external side 41 of the electrode 5 is covered by soft cosmetically appealing molded foam or elastomer as seen in FIG. 3. Once the battery 22 is depleted, the entire electrode-battery assembly 18 can be disposed of or replaced. The unique advantage provided by the electrode-battery assembly 18 is its ability to combine both the electrodes 5 and batteries 22 in one separate housing thereby supporting different battery technologies. Therefore, the housing 2 can be produced in large quantities regardless of the type of battery configuration utilized as long as the housing 2 is designed with the requirement that two 1.5 batteries, one on each electrode, or a single 3 volt battery are to be provided to it. When multiple batteries are used a series connector may be optionally placed between a positive pole of one battery and a negative pole of the other battery to increase the voltage. If increased voltage is not desired the batteries can be wired in parallel by connecting like poles to increase the available amperage One of skill in the art will understand how to modify the circuit in order to properly wire plural batteries.

Also accompanying the TENS device, with which the electrode-battery assembly is a part, is a docking station (not shown) which can be used for recharging the TENS device when it is not in use. The docking station provides the patient flexibility in selecting the appropriate battery configuration given varying factors including cost, size and time of use. Many docking station configurations exist, however each contains battery contacts for battery 22 connection and electrode contacts for electrode 5 connections.

The typical docking station configuration comprises button cell or cylindrical cell batteries; a housing with mating features to the electronics module 20, and which houses the batteries; a circuit board with battery contacts for connection to the electronics module 20 and the batteries; and a voltage regulator and female jacks for accepting lead wires from the electrodes 5. In an alternate embodiment of the docking station described above, mechanical clamping means are used to attach electrode conductive material directly to the circuit board, as opposed to lead wires. In yet another embodiment of the docking station, the batteries are placed directly over the electrodes 5 as an assembly of the electrodes 5. This can be accomplished either with or without the use of lead wires.

Of particular relevance here, another docking station configuration comprises a lithium polymer battery assembled as a flexible layer uniquely integrated as part of the electrode-battery assembly 18. Replacing the traditional batteries 22 of the traditional electrode-battery assembly 18 described above is a lithium polymer battery assembled as a flexible lithium-ion polymer battery layer, and an insulation layer. The advantage of this assembly 18 is its low-profile design that makes the batteries virtually invisible to the user. The assembly 18 is lightweight, flexible and has superior conformability and rechargeability features. The disposable electrodes 5 can be removed and replaced by peeling the durable lithium polymer layer away from the insulation layer.

Any of the above docking station configurations can be used as an integral assembly to a standard splint, bandage, manufactured brace, or cast 36.

Electronics for a standard splint, bandage, manufactured brace, or cast would attach and detach from the electrode-battery assembly and offer different stimulation modes. In this embodiment, the electrode-battery assembly would remain disposable and the electronics module reusable. Incidentally, in this embodiment the device can be operated with or without a remote controller.

Electrical connection and current flow between the electronics pack and the electrode assembly to the patient is shown in FIG. 4A and established as follows: Battery positive connection 49 is made through positive battery conductor 49 and connects to the positive input pin of the electronics pack (not shown) at contact 52. Battery negative 50 is made through conductor 53 and is insulated from the positive side of the battery and positive conductor by insulator 54. The negative battery conductor is connected to the negative input pin of the electronics pack at negative contact 55. Configuration resistor 56 establishes part of a voltage divider network inside the electronics pack and makes connection from the positive conductor 52 of the electrode through said resistor 56 and onto resistor conductor 57 where it is then connected to the electronics pack configuration resistor input pin at contact 58. Stimulation current from the electronics pack is conducted from the stimulation output pins of the electronics pack into and out of electrode contacts 59 and 59′. Current flow can be conducted in either direction at the will of the electronics pack, therefore direction of flow is indeterminate. For sake of illustration, the path of conduction for current flow starts at contact 59′, goes through conductor 60′, passes down through the base substrate 61 through a conductive via 62′, through conductor 63′, into the conductive hydrogel or adhesive 64′ and into or through the patient, returning through conductive hydrogel 64, through conductor 63 up through the base substrate 61 through conductive via 62, through conductor 60 and back into the electronics pack through connection at contact 59. Conductors 63 and 63′ are insulated from the patient by insulator layers 65 and 65′.

The conductive via are created by forming a region of at least one small hole through the base layer. The holes may be any number and any size but are preferably laser cut to the diameter of the laser beam and are sufficient in number such that they are capable of carrying the required current. These holes are then filled with either a conductive ink or have conductive material deposited thereon such that the hole becomes conductive from one side of the substrate to the other. Small laser drilled holes will aid in the manufacturing process because they will draw the conductor material into the holes through capillary action. The number of holes can readily be determined by calculating the cross section area of the conductor being used that is required to carry the intended current and dividing it by the cross sectional area of holes. Alternatively but less preferred, it is possible to use standard means of conducting current from one side to the other, such but not limited to conductive pins. Such pins would be inserted before printing to allow the conductive inks to bond to the pins.

The configuration resistor 56 is used to program the current amplitude of the attached TENS device. When a patient connects the TENS device to the electrode and turns on the TENS device, the resistance of the electrode is read by the TENS device and establishes a specific voltage based on the resistance. This voltage is read by the processor in the TENS device and is then correlated to a table embedded in the software which establishes allowed modes of operation and the intensity limits. The TENS unit may allow some user settable variation in current strength but such current will be effectively capped by the resistance in the electrode. Having such a self configuring device will eliminate or reduce concerns that a patient can deliver too high or an otherwise inappropriate voltage. The configuration resistor can be a traditional resistor inserted into the circuit or more preferably imprinted using resistive ink. Similarly other control mechanisms can be introduced. The electrode can contain a computer chip or RFID device which is read by the TENS device and the setting adjusted in accordance with the instructions on the chip or RFID device.

The conductive medium can be any form of electrical conductor capable of conducting current through the base substrate from the electrode contacts to the electrode. Electrically conductive elements can be inserted through the substrate, electrically conductive adhesives can be used or printed traces can be left exposed to allow for direct electrical contact with the contact pins of the electronics package.

The electrode assembly design embodied herein is preferably a unitary design in which the electrodes are connected together by the non-conductive substrate. The non conductive substrate is patterned to aid in placement of the electrodes around the desired part of a patient's anatomy. The positioning of the electrodes on the non-conductive substrate is also determined by where the electrode assembly is to be placed on the patient.

The electrode can be constructed using conventional continuous process printing techniques. Such techniques are known in the art and rely on a non-conductive substrate comprising a polymer or other non-conductive material upon which conductive traces and dielectric insulating layers are sequentially printed in order to form electrical contact points for the interface to the TENS device, the batteries and the configuration resistor and the conductive hydrogel. After printing, printed layers and the battery or batteries are sealed under a thin non-conductive polymer film. Alternatively the circuit can be folded such that the non-conductive polymer base layer serves as a top cover. The polymer film is configured to have openings over the contact points for the electronics for the purpose of making electrical connection therewith. The TENS device preferably maintains electrical contact with the electrode by means of stamped metal spring contacts or machined spring contact pins on the TENS device which make contact with the electrode. It is also readily apparent that any suitable means for maintaining an electrical connection may be used. The electronics enclosure is preferably attached to the electrode by means of a mating latching mechanism contained on the electronics and the electrode such that when the electronics are inserted on the electrode in the proper orientation, the electrode would fasten the electronics. Other suitable means for attachment would include but are not limited to hook and loop fasteners, snaps, flaps, tapes, pockets created on the electrode and/or adhesives provided they have sufficient strength to securely hold the electronics in place. Access to the printed conductive traces and hydrogel on the bottom side of the substrate layer is accomplished through openings cut in the substrate and filled with conductive material or by a separate conductive substrate laminated to the main substrate in order to make contact with both the top and bottom of the main substrate.

The electrode 64 and the electrode conductors can be formed by applying electrical conductors on a non-conductive flexible substrate. In a preferred embodiment the substrate is polyester sheeting such as is sold under the trademark Mylar. However, other flexible substrates will also work if they have suitable mechanical and non-conductive properties. A preferred conductor for this application is silver/silver chloride epoxy ink. One conductor is printed on the substrate for each electrode. At least two electrodes are required to be present for a TENS device to operate in this embodiment.

It will be appreciated that the programmable electrode can take other forms other than a combination battery-electrode assembly. For example, the resistance can be made part of a conventional wire set wherein a resistor wire is used to make a connection between two terminals on the TENS device and program it. In such instance, the resistor would induce a specific voltage in the TENS unit which would be correlated to a reference table embedded in the software to identify the appropriate current settings for the patient. Such a wire set would allow for the use of disposable patch electrodes while still maintaining the user friendliness of a self programming device. Additionally, the battery or other power source can be contained in the TENS unit. In such instance, the electrode assembly would omit the battery and just have conductors between the electrodes and the TENS unit.

Although in this described embodiment the electrodes and traces are silk screened on a substrate, in alternative embodiments, the flexible electrode array can be produced by any process that is operative to deposit or print a specifically defined pattern of conductive materials on a flexible sheet. Examples of such other processes includes flexographic printing with conductive inks. In other embodiments subtractive methods can be used such as chemical etching of aluminum or copper on clear polyester.

In addition, rather than insulating trace lines with non-conductive inks, other embodiments may include a non-conductive overlay sheet for insulating the printed trace lines. Such an overlay would leave the electrodes and connector ends exposed by including a plurality of apertures in the overlay which coincide with the printed electrodes and connector ends.

One advantage of printing both the electrode and the traces on a clear flexible plastic substrate such as polyester sheet is the reduction in the cost associated with manufacturing the flexible electrode array. The lower cost enables the flexible electrode array to become a disposable part in the TENS system; thus, eliminating the need to clean electrodes between uses of the system. In addition, using a transparent substrate such as a polyester sheet, aids in the accurate positioning of the electrodes by allowing a clinician to see the underlying anatomy of the patient through the flexible electrode array. Thus, after a clinician has marked the locations of vertebra on a patients back, the clinician can precisely position the center column of the printed electrodes over these markings.

Another advantage of using a polyester substrate such as Mylar® is that polyester film is a material that is both tear resistant and sufficiently flexible to conform to the general shape of a patient's back. Further, the present invention achieves increased flexibility and extensibility in the design of the flexible electrode array by including a plurality of strategic slits in the substrate to make the flexible electrode array extensible (stretchy) in between electrodes. This enables the flexible electrode array to stretch or compress in three directions (horizontal, vertical, and diagonal).

Accordingly, it will be understood that the preferred embodiment of the present invention has been disclosed by way of example and that other modifications and alterations may occur to those skilled in the art without departing from the scope and spirit of the appended claims.

We claim:

1. An electrode-battery assembly to be used in a miniature wireless transcutaneous electrical neuro or muscular-stimulation unit comprising:
a plurality of electrodes each having an internal and external side; a at least one battery having a positive and negative pole; a flexible conductive carrier with a hydrogel which carries current to a pain site or other area on a user's body via said electrodes; conductive film comprised of three current carrier runners wherein two of said runners are in direct contact with each of said positive and negative poles of said battery and a third said runner is in direct contact with said hydrogel; and a mechanical battery clip which secures said runners to said positive and negative battery poles.

2. The electrode-battery assembly of claim 1 wherein said electrode-battery assembly is disposable and can be replaced upon depletion of said battery.

3. The electrode-battery assembly of claim 1 wherein said conductive film is comprised of a silver alloy film, silver ink channel, gold, graphite, copper or some other flexible low impedance material.

4. The electrode-battery assembly of claim 1 wherein said external side of said electrode is covered by a molded cover comprised of a cosmetically appealing molded foam or elastomer.

5. An electrode-battery assembly to be used in a miniature wireless transcutaneous electrical neuro or muscular-stimulation unit comprising:
a plurality of electrodes for conducting a current to a patient;
a flexible non-conductive substrate having top and bottom surfaces, wherein a surface of the substrate has a means for attaching a transcutaneous electrical neuro or muscular-stimulation unit;
at least one battery having a positive and negative pole disposed on a surface of the non-conductive substrate,
a means for retaining the battery on the electrode-battery assembly comprising an insulated material;
an electrical conductor comprised of at least one insulated current carrier runners disposed on the top surface of said flexible non-conductive substrate wherein at least one runner makes electrical contact between a pole of the battery and the transcutaneous electrical neuro or muscular-stimulation unit device,
a plurality of electrode conductors comprised of insulated conductive runners also disposed on the top surface of the substrate wherein said runners make electrical contact between the transcutaneous electrical neuro or muscular-stimulation unit and one of said electrodes per runner, and
a means for conducting an electrical current through the non-conductive substrate from the electrode conductor to the electrode.

6. The electrode-battery assembly of claim 5 wherein said conductor is comprised of a silver alloy film, silver ink channel, or some other flexible low impedance material.

7. The electrode-battery assembly of claim 5 wherein said external side of said assembly is covered by a cover.

8. The electrode-battery assembly of claim 5 wherein the means for conducting an electrical current through the non-conductive substrate from the electrode conductor to the electrode is through a conductive material passing through the non-conductive substrate.

9. The electrode-battery assembly of claim 8 wherein the conductive material is a conducive via.

10. The electrode-battery assembly of claim 9 wherein said conductive via are formed by making at least one hole through the conductive substrate and applying an electrically conductive substance to the hole.

11. The electrode-battery assembly of claim 5 wherein the conductive runners are comprised of a silver, gold, copper or graphite containing ink or some other flexible low impedance material.

12. The electrode-battery assembly of claim 5 wherein the conductive runners are comprised of a silver epoxy ink.

13. The electrode-battery assembly of claim 5 wherein a hydrogel is applied to the electrodes.

14. A method of providing electrical stimulation therapy to a patient in need comprising:
a. attaching a unitary electrode-battery assembly to an electronic device consisting of a transcutaneous electrical neuro or muscular-stimulation unit;
b. programming the transcutaneous electrical neuro or muscular-stimulation unit
c. attaching the transcutaneous electrical neuro or muscular-stimulation unit and electrode battery assembly to the patient, and
d. starting the transcutaneous electrical neuro or muscular-stimulation unit.

15. The method of claim 14, wherein the unitary electrode-battery assembly has at least two electrodes of opposite polarity in contact with the patient.

16. The method of claim 14, wherein the electronic device is a transcutaneous electrical neuro stimulation unit or a transcutaneous electrical muscular stimulation unit.

17. The method of claim 9, wherein a hydrogel is applied to the electrodes prior to attachment to the patient.

18. The electrode-battery assembly of claim 1 further comprising:
a non-conductive top layer affixed to the external side of the electrode having at least one cutout thereon to allow a TENS device to make electrical contact means for conducting current to and from the conductive traces to the TENS device.

19. The electrode-battery assembly of claim 1 wherein an electrically conductive adhesive is used to conduct current to the patient instead of a hydrogel.

20. The electrode assembly of claim 1 further comprising a means for programming a transcutaneous electrical neuro or muscular-stimulation unit.

21. The electrode assembly of claim 20, wherein the means for programming the transcutaneous electrical neuro or muscular-stimulation unit comprises a resistor, resistive ink or an RFID chip.
